(19) United States
(12) Patent Application Publication
(10) Pub. No.: US 2015/0110713 A1
(43) Pub. Date: Apr. 23, 2015

(54) METHOD AND COMPOSITION FOR TREATING CANCER

(71) Applicants: Anthony Manganaro, Columbia, MD (US); Karen Rockwell, Malden, MA (US)

(72) Inventors: Anthony Manganaro, Columbia, MD (US); Karen Rockwell, Malden, MA (US)

(21) Appl. No.: 14/579,176

(22) Filed: Dec. 22, 2014

Related U.S. Application Data
(63) Continuation of application No. 13/348,032, filed on Jan. 11, 2012, now abandoned.
Application No. 13/348,032 is a continuation of application No. 12/044,761, filed on Mar. 7, 2008, now abandoned.
(60) Provisional application No. 60/905,902, filed on Mar. 9, 2007.

(51) Int. Cl. A61K 31/375; A61K 31/454; A61K 33/24; A61K 31/17; A61K 33/36; A61K 31/404; A61K 31/337; A61K 51/12; A61K 51/04; A61K 51/08; A61K 38/14; A61K 31/711; A61K 31/7105; A61K 49/00; A61K 47/48

(52) U.S. Cl. CPC A61K 31/375; A61K 47/481; A61K 47/48092; A61K 31/454; A61K 33/24; A61K 31/17; A61K 33/36; A61K 31/404; A61K 31/337; A61K 51/12; A61K 51/04; A61K 51/08; A61K 38/14; A61K 31/711; A61K 31/7105; A61K 49/0002
USPC 424/1.21; 514/474; 514/323; 424/649; 514/588; 424/623; 424/450; 424/490; 514/414; 514/449; 424/1.65; 424/1.29; 424/1.69; 514/19.3; 514/19.4; 514/19.5; 514/44 R; 514/44 A; 424/9.1

(57) ABSTRACT

A method for treating cancer, preventing cancer or delaying the progression of a cancer in an animal or human comprising the step of: administering to the animal or the human having a cancer a composition in an amount effective to treat cancer, prevent cancer or delay the progression of cancer in the animal or the human. The composition comprises a pharmaceutically acceptable excipient, and ascorbate which is joined to a carrier structure containing an anti-cancer active agent, said carrier structure being capable of releasing the anti-cancer agent in the presence of a reactive oxygen species.

### CROSS-REFERENCE TO RELATED APPLICATIONS

This application is a continuation of U.S. patent application Ser. No. 13/348,032, filed on Jan. 11, 2012, which is a continuation of U.S. patent application Ser. No. 12/044,761, filed on Mar. 7, 2008, which in turn is based on U.S. Provisional Patent Application No. 60/905,902, filed on Mar. 9, 2007, all of which are incorporated herein by reference in their entireties.

### BACKGROUND OF THE INVENTION

1. Field of the Invention

The present invention relates to methods and compositions for the effective delivery of anti-cancer agents to cancer tumors in patients. This application claims benefit of Provisional Application 60/905,902 filed Mar. 9, 2007, which is incorporated herein in its entirety by reference.

2. Description of the Related Art

Cancer is a genetic disease and in most cases involves mutations in one or more genes. There are believed to be around 60,000 genes in the human genome. Cancer cells exploit hundreds of gene products and pathways to enable or enhance their malignancy. Although understanding the cancer-type specific pathways that enhance malignant progression is important and leads to new powerful treatments, all cancer researchers dream of finding a common way to kill many types of cancer cells while leaving normal, critical tissues with minimal damage. One benefit of certain embodiments of the present invention is that they use metabolic traits common to many cancer and solid tumor types to target cancer cells for treatment while sparing normal tissue from the potentially toxic side effects of treatment with anti-cancer agents.

The delivery of cytotoxic or chemotherapeutic agents to the site of a solid tumor is highly desired because systemic administration of these agents can result in killing not only the tumor cells, but also normal cells within the body. This toxicity to normal cells limits the dose of the cytotoxic agents and thus reduces their therapeutic potential. However, in some instances the administered agent has no intrinsic activity, but is converted in vivo at the appropriate time or place to the active drug. Such analogues are referred to as pro-drugs and are used extensively in medicine. Conversion of the pro-drug to the active form can take place by a number of mechanisms depending, for example, on changes of pH, oxygen tension, temperature or salt concentration or by spontaneous decomposition of the drug or internal ring opening or cyclization.

Targeted drug delivery has been tried with antibodies and linked pro-drugs. WO 88/07378 describes a two-component system and its therapeutic uses. A first component comprises an antibody fragment capable of binding with a tumor-associated antigen and an enzyme capable of converting a pro-drug into a cytotoxic drug, and a second component which is a pro-drug which is capable of conversion to a cytotoxic drug. This general system, which is often referred to as “antibody-directed enzyme pro-drug therapy” (ADEPT), is also described in relation to specific enzymes and pro-drugs in EP 0 302 473 and WO 91/11201.

Nanometer-scale drug carriers such as liposomes and polymersomes have been developed to deliver drugs to disease sites, and are increasingly common in clinical use. The principals of nanocarrier design and biological interactions are increasingly well understood, allowing tailored design of nanocarriers with specific drug delivery, targeting, and release characteristics.

Yet, despite extensive research and investigation of these and other systems, there is still an urgent need for a treatment system which simultaneously attacks cancer cells but does not damage normal cells.

### SUMMARY OF THE INVENTION

The present invention is a composition for administration, and a method of administering such a composition, to a cancer patient. The composition contains the pharmaceutically acceptable agent, ascorbate, which is linked to a carrier structure containing an anti-cancer active agent, the carrier structure being capable of delivering the anti-cancer active agent in the presence of a reactive oxygen species. In some preferred embodiments, the structure is a nanocarrier such as a polymersome or liposome. The reactive oxygen species is a preferably superoxide, hydrogen peroxide, or a combination thereof.

It is believed that the administration of the composition of the present invention is effective in treating, preventing or delaying the progression of, cancer. The mechanism by which the composition of the present invention functions is not yet known, but is believed to involve the respective chemical, pharmacological, and physical properties of ascorbate toward solid tumors, tumor cells, and their surrounding microevironments.

Ascorbate has a molecular structure similar to sugars. This similarity in structure is believed to allow the oxidized form of ascorbate, called dehydroascorbate, to be transported into cells by the glucose transporters known as GLUT's, typically GLUT-1, GLUT-3, GLUT-4. These glucose transporters are found on virtually every cell in the body, and many cancer cells over-express them. Up to 300,000 GLUT transporters may be found on a cell surface, at a density of up to 2000 transporters per square micrometer of cell surface.

Dehydroascorbate is rarely formed in normal tissues, and is short lived. In tumor tissues, however, DHAA is generated in abnormal amounts through the action of the high levels of superoxide anion produced by the tumor and by its surrounding support tissues called the stroma. This tumor-microenvironmental production of DHAA is believed to allow tumor cells to accumulate ascorbate in large quantities. Normal cells do not. Such accumulation is a symptom of the cancer cells' altered metabolism. Increased oxidation of ascorbate on the surface of nanocarriers in the tumor microenvironment would increase concentrations of DHAA on the surface of nanocarriers. DHAA on the surface of a nanocarrier can bind to glucose receptors on cell surfaces, thereby allowing enhanced nanocarrier associations with tumor cells.

Cancer cells are susceptible to death caused by high concentrations of extracellular ascorbate, which produces hydrogen peroxide in tissues. Normal cells are much less susceptible. This susceptibility gives another theoretical mechanism by which the composition and method of the present invention may have anti-tumor effectiveness; the production of hydrogen peroxide from the ascorbate nanocarrier would provide tumor-specific cytotoxicity. Because of the poor circulation in many tumors, the peroxide produced by the ascorbate of the disclosed composition accumulates, providing localized anti-tumor therapy even before the release of drug agent from the nanocarrier of the disclosed composition.

Tumor cells are more sensitive to death caused by hydrogen peroxide than normal cells are. This is in part because normal cells have ample levels of redox regulating molecules, enzymes, and other metabolic factors, whereas cancer cells tend not to. Catalase, peroxidases, and other Reactive Oxygen Species (ROS)-detoxifying enzymes help prevent ROS accumulation in normal cells by keeping the concentrations of ROS inside and outside the cell safe. In tumor cells, ROS and RNS from numerous sources inside and outside of the cells strain the cellular detoxification mechanisms, leading to oxidative stress. Thus, increased ROS from extracellular peroxide generation from ascorbate can overwhelm the tumor cells and lead to cellular damage. Normal cells are not overwhelmed by the additional peroxide generated by extracellular ascorbate.

The reactive oxygen species created in or in proximity to the cancer cells can be used to trigger delivery of the anti-cancer agent from the carrier structure within or in proximity to the cancer cell.

The ascorbate in the surface of the present invention could provide high local concentrations of ascorbate to act as a pro-oxidant, leading to the production of hydrogen peroxide in tumor tissues. The poor perfusion within tumor tissues would allow local accumulation of the ascorbate-generated peroxide, which could directly damage tumor cells. This local accumulation of hydrogen peroxide could enhance delivery of drug from the nanocarrier.

It is recognized that reactive oxygen species, such as hydrogen peroxide, in sufficient amounts, may be harmful to the human body. Unlike tumor tissues, normal tissues and fluids are capable of neutralizing excess ROS through their robust enzymatic defenses such as superoxide dismutase, catylase, and others, and would thus avoiding accumulation of cell-damaging concentrations of hydrogen peroxide or other reactive oxygen species produced by the nanocarriers.

Ascorbate also has collateral benefits, including enhancement of the immune system. There is also evidence that ascorbate administered in conjunction with chemotherapy drugs has sensitized the cancer cells to those drugs, thereby promoting their effectiveness.

### DETAILED DESCRIPTION

The carrier structure of the composition is desirably selected from among those known in the art, including but not limited to those disclosed in U.S. Patent Application Nos. 2004/0062778, 2004/0109894, 2005/0003016, 2005/0031544, 2005/0048110, 2005/0180922, 2005/0191359, 2006/0240092, 2005/0244504, 2005/0265961, 2006/0165810 and 2006/0280795. Each of these applications is incorporated herein by reference in its entirety by reference.

Preferably, the nanocarrier type is a member selected from the group consisting of liposomes, stabilized liposomes, cross-linked liposomes, polymersomes, stabilized polymersomes, cross-linked polymersomes, micelles, stabilized micelles, cross-linked micelles, dendrimers, nanoparticles, protein-based carrier, aptamers, nanoshells, chitin-based carrier, gels, and colloids.

Most preferably, the nanocarrier is a pharmaceutically acceptable nanocarrier composition. Lipids used in liposomal nanocarrier formulations are preferably members selected from the group consisting of phosphatidylcholines, phosphatidylethanolamines, phosphatidic acids, phosphatidylserines, phosphatidylglycerols, cardiolipins, poly(ethylene glycol) lipid conjugates, sphingomyelins, cationic lipids, trioctanoin, triolein, dioctanoyl glycerol, cholesterol, and dioleoyl-glutaric acid.

Components of polymer-based nanocarriers are preferably members selected from the group consisting of block polymers, poly(ethylene glyol), N-(2-hydroxypropyl)methacrylamide, poly(L-lysine), poly(L-glutamic acid), poly(lactic-co-glycolic acid), polylactide, poly(propylene sulfide)poly(alkyl cyanoacrylate), poly(ethylene oxide), poly(epsilon-caprolactone), poly(butyl cyanoacrylate, distearoylphoshatidylethanolamine, polyethyleneimide.

The nanocarrier of the present invention can contain components that are sensitive to ROS and RNS. The characteristic reactions of ROS and RNS can be used to alter carrier components in order to cause alterations of carrier characteristics preferably selected from the group consisting of changes to hydrophobic/hydrophilic balance of nanocarrier components, disintegration of nanocarrier structure, formation of smaller particles, enhancement of membrane fusion with target cells, shedding of components or component pieces from the nanocarrier. Components sensitive to ROS and RNS are preferably members selected from the group consisting of poly(propylene sulfide) blocks ,peroxide-sensitive lipids, and triblock polymer PEO-(p)-PPS) where PEO is a very long PEG chain and PPS is poly(propylene sulfide). Oxidation of the hydrophobic propylene sulfide to hydrophilic poly(sulfoxides) and poly(sulfones) results in formation of soluble oxidized copolymer.

The nanocarrier of the present invention can contain components that are sensitive to low pH found outside of and inside of tumor cells. Components sensitive to low pH of tumors could produce nanocarrier changes preferably chosen from the group consisting of altering the hydrophobic/hydrophilic balance of nanocarrier components, disintegration of nanocarrier structure, formation of smaller particles, enhancement of membrane fusion with target cells formation of lipid penetrating micelles, shedding of components from the nanocarrier, and many other changes. The pH-sensitive component is preferably a member selected from the group consisting of poly (Beta-Amino Ester), poly (L-histidine), poly(DL lactide), poly(vinyl alcohol), sulfonamide-modified polymers,PEI, N-isopropylacrylamide, and polyacrylamide.

Components sensitive to low pH of endosomes could produce carrier changes preferably chosen from the group consisting of altering the hydrophobic/hydrophilic balance of nanocarrier components, disintegration of nanocarrier structure, formation of smaller particles, enhancement of drug escape into the cytoplasm of target cells, shedding of components from the nanocarrier, formation of lipid penetrating micelles. And endosomal rupture. The pH-sensitive component is preferably chosen from the group consisting of poly(L-lactide), polycaprolactone , poly(Beta-Amino Ester), polylactic acid, poly(DL lactide), poly(Beta-Amino Ester), poly(L-histidine), poly(vinyl alcohol), N-isopropylacrylamide, and polyacrylamide, HPMA N-(2-hydroxypropyl)methacrylamide copolymer. PH sensitive linkages can be used to release an active agent or carrier component in low pH environments, and are preferably chosen from the group consisting of dimethyl maleic anhydride, cis-aconityl, and hydrazone linkages.

The nanocarrier of the present invention can have cell targeting components preferably selected from the group consisting of antibodies,ligands, cell penetrating peptides, cationic peptides, TAT sequences, nuclear localization signals, mitochondrial localization signals, release peptides for endosomal destabilization

The carrier structure of some embodiments of the present invention may also be micelles created from monomers having at one end ascorbate head group, the other end being capable of forming an acid sensitive bond to the active agent of the present invention. The micelles are capable of being triggered by a reactive oxygen species to release the active agent.

The carrier structure of some embodiments of the present invention may be in the form of a nanocarrier, the core comprising an active agent. Surrounding or effectively surrounding the active agent core may be an intermediate layer which is designed to open or activate in response to a reactive oxygen species, preferably peroxide or superoxide anions. Surrounding or effectively surrounding the intermediate layer is an outer layer which contains ascorbate. The ascorbate is in an amount sufficient such that upon administration to the cancer patient it generates a reactive oxygen species. The ascorbate also may help the nano-particles to adhere to or be retained in the tumor cells through interaction with those cells' glucose transporters. The outer layer may contain peptides, such as cationic peptides, which are believed to promote mediation of the inventive particles into cancer cells.

The carrier structure may include surfactants, where they modify the particle surface characteristics. The surfactant is selected from the group consisting of anionic surfactants, cationic surfactants, zwitterionic surfactants, nonionic surfactants, surface active biological modifiers and combinations thereof.

Examples of suitable materials for the carrier structure of the present invention include the multi-block copolymers disclosed in U.S. Application No. 2003/0059906, the pH-triggerable particles disclosed in U.S. Application No. 2005/0244504, the poly(β-amino esters) disclosed in U.S. Application No. 2005/0265961, the multi-block copolymers disclosed in U.S. Application No. 2006/0240092, and the polyoxyethylene-based polymersomes disclosed in U.S. Application No. 2005/0003016, the amphiphilic block copolymers and self-assembled polymer aggregates disclosed in U.S. Pat. No. 6,569,528, the polymersome vesicles disclosed in U.S. Pat. No. 6,835,394, and the block copolymers disclosed in U.S. Pat. No. 7,132,475, each of these documents being incorporated herein by reference in its entirety.

The active agent of the present invention is preferably chosen from the group consisting of 5-FU, ceramide, cisplatin, cyclophosphamide, flutamide, imatinib, levamisole, methotrexate, motexafin gadolinium, oxaliplatin, paclitaxel, tamoxifen, taxol, topotecan, and vinblastine. Antineoplastic quinones may be used, for instance, daunorubicin, diaziridinylbenzoquinone, doxorubicin and mitomycin C. Also possible are carmustine, chlorambucil, denileukin diftitox, ibritumomab tiuxetan, lomustine, and tositumomab (such as for the treatment of lymphoma); docetaxel, fulvestrant, pamidronate, thotepa, and trastuzumab, (such as for the treatment of breast cancer); dacarbazine and interferon (such as for the treatment of melanoma); cisplatin, etoposide phosphate, ifosfamide, vinblastine, (such as for t he treatment of testicular cancer). Another agent is arsenic trioxide (As₂O₃; ATO) which is effective in the treatment of relapsed acute promyelocytic leukemia (APL), inducing partial differentiation and promoting apoptosis of malignantpromyelocytes. Antiangiogenics and immune modulating treatments are excellent options for ascorbate nanocarrier cargo. Such treatments include thalidomide, lenalidomide, protein kinase inhibitors, and others. Sunitinib (Sutent) may be an active agent, used for treatment of gastrointestinal stromal tumors. It is believed that Sunitinib inhibits receptor tyrosine kinases (RTK's) which used by certain cancers such as RCC to drive tumor growth. The active agent may also be one of those disclosed in U.S. Application No. 0070032534, now pending, which is incorporated herein by reference in its entirety.

Drugs that target critical molecules in the hypoxia-induced cellular adaptation are potential cargo for the drug delivery system of this invention. These include, for example, drugs that inhibit the activity of HIF-1, a gene regulator which induces numerous proteins to be made which help normal cells to survive transient low oxygen. Tumors exploit this pathway to survive and grow in prolonged hypoxia and also to grow more aggressively in the presence of oxygen. The proteins hypoxia inducible factor la, carbonic anhydrase IX, vascular endothelial growth factor, and other members of the hypoxia-induced gene family may also be used as targets for the active agent. These proteins are useful targets for cancer drug therapy because many cancers use these hypoxic responses to allow continued growth under highly stressed conditions. The von Hippel-Lindau tumor suppressor gene codes for a protein which normally helps the cell degrade another regulator; HIF-la (alpha). The HIF-1 gene regulator induces production of such targets as GLUT-land GLUT-3 glucose transporters, VEGF angiogenisis-promoting growth factor, the TGF and IGF growth factors, CA IX , NAD(P)H oxidases and ROS. Genetic information allowing the production of von Hippel-Landau tumor suppressor protein in tumor cells could be introduced to tumor cells as a means of normalizing their HIF-1 regulation, using this drug carrier system The genes induced by hypoxia often enhance malignant progression of tumor cells and result in treatment resistance. The ascorbate targeting in this proposed system can exploit hypoxia-induced gene patterns to enhance tumor treatment.

Hypoxic gene regulation, TNFalpha, VEGF, IGF, and other tumor factors can enhance the effectiveness of the nanocarriers of the present invention through several mechanisms. Leaky, convoluted tumor vasculature symptomatic of angiogenic factor exposure can allow improved nanoparticle accumulation within the perivascular spaces of the tumor. TNFalpha and hypoxic signaling pathways can lead to increased ROS production, which can increase the conversion of ascorbic acid to dehydroascorbic acid. In addition, hypoxic signaling pathways increase expression of glucose transporters to which dehydroascorbic acid can bind.

The cancers to be treated, prevented or delayed with the method and composition of the present invention are preferably chosen from the group consisting of Hodgkin's Disease, Non-Hodgkin's Lymphoma, neuroblastoma, blood cancers, brain cancer, breast cancer, ovarian cancer, liver cancer, pancreatic cancer, lung cancer, rhabdomyosarcoma, primary thrombocytosis, primary macroglobulinemia, small-cell lung tumors, non-small-cell lung tumors, primary brain tumors, stomach cancer, renal cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, myeloid leukemia, small tissue sarcomas, osteosarcomas, and prostate cancer.

The ascorbate of the present invention is not particularly limited with respect to its form. Ascorbate is preferably linked at the 2 and/or 6 position to lipid or polymer nanocarrier components.

The ascorbate of the present invention may be linked to the carrier structure via a covalent bond, such as by a sulfur atom, an oxygen atom or a hydrocarbon linking group. These linkers are often, but not always, sensitive to pH and oxidation to mediate release of the active agent. Possible linkers include dimethyl maleic anhydride, cis-aconityl, and hydrazone which are sensitive to change in pH values. Also possible are peptide sequences, especially cationic peptide sequences, which cleavable by common proteases, such as cathepsin-cleaved peptide sequence GFLG.

The composition of the present invention should be administered in an amount sufficient to impart a therapeutic effect to the patient with respect to the cancer. The dose of the invention may be determined, in part, by the volume of the tumor to be treated, because the density of microparticle accumulation in the tumor tissue will determine effectiveness. Drug or particle concentrations in the blood and critical normal tissues will limit upper doses, and will vary depending on the anti-cancer agent chosen as cargo. Generally speaking, the composition could theoretically be generated that has concentrations of should be administered to the patient in an amount which results in a tumor concentration of ascorbate above 1000 μmol/L. Alternatively, the ascorbate level may be 20 mM or greater, to a level which does not harm the patient. As a further alternative, the composition of the present invention may be administered in a therapeutically effective amount.

In some embodiments of the present invention, there may be 100,000 to 1,000,000 ascorbate groups per carrier particle. Other embodiments may use higher or lower numbates of ascorbate per carrier structure. The number of ascorbate groups per nanocarrier will depend on the concentration of ascorbate desired as well as the size of the carrier structure. In some embodiments , the nanocarriers may have multiple ascorbate molecules per component strand. Drug incorporation into carrier particles varies widely, but drug loading of 4-20% weight per volume is likely a suitable common range. Drug loading depends on carrier lumen size, drug size, interactions between lumen and drug, and the method of loading the carriers.

One might model a carrier particle volume as a cube for simplicity. A nanocarrier of diameter 100 nm fits in a cube 10⁻⁷ meters on a side, having a volume of 10⁻²¹ cubic meters. One liter takes up a cube 0.1 meters on a side (0.001 cubic meters in volume). Therefore the volume of a nanocarrier is ˜10⁻¹⁸ liters. Since a one molar solution contains 6.02×10²³ molecules per liter, a 100nm particle composed of 1M ascorbate contains 6×10⁵ molecules of ascorbate.

If a carrier is composed of 100,000 units and each polymer or lipid string contained just one ascorbate group, that would result in a 160 mM solution strength equivalent in the 10-18 liter carrier volume. If this level is too high, mixing polymers some of which contain ascorbate and some of, which do not, could be considered. Conversely, if the ascorbate can be shielded from casual contact with normal vasculature and tissues, increasing numbers of ascorbate groups per strand to 6 or more could attain local concentrations of nearly 1M under very limited circumstances—such as when a particle has adhered to a cell surface. This stresses the importance of shielding some of the ascorbate groups within the brush coat of the carrier to limit active agent interactions with vascular tissues and potential oxidative effects on non-targeted cells. Such shielding could be ROS- or acid-sensitive, allowing the shielding to be shed in the tumor site to make the carrier less stealthy and more likely to adhere to cell surfaces once the nanocarrier enters a tumor.

More directly, 10⁵ to 10⁶ ascorbate molecules per carrier is attainable. Some fraction of these will produce peroxide in the tumor tissue. A small amount will react as the carrier flows through the blood, but the erythrocytes' catalase will scavenge the peroxide produced. After entering the tumor through leaky vasculature, the carrier will generate peroxide as it diffuses through the tumor interstitial fluids. Ascorbate will convert to dehydroascorbic acid upon exposure to the microenvironmental superoxide anion. It was reported that a 20 millimolar ascorbate solution in vitro generated 150 micromolar H₂O₂, a level which is believed to be toxic to tumor cells and not normal cells. The density of ascorbate on the particle surface will need to be titrated so that it is toxic to tumor cells and not toxic to normal cells. It may be that only 10% of polymer strands in a carrier design should contain ascorbate to avoid toxicity. Alternatively, some designs that shield the ascorbate groups may allow the use of multiple ascorbate groups on some or all polymer strands and yet still avoid normal tissue toxicity.

The nanocarrier of the present invention can also use ascorbate and dehydroascorbate to cause localized glutathione depletion within a cell. GSH is a central antioxidant and reducing agent in cellular metabolism. As such, GSH has roles in diverse cellular functions. GSH can react directly with DHAA, without enzymes, which contributes to the coupling between the ascorbate and GSH redox regulation pathways. More specifically, GSH is a cofactor for glutathione peroxidase and other oxidative stress-reducing enzymes, scavenges hydroxyl radical and singlet oxygen, and helps regenerate ascorbate and vitamin E to active forms . Glutathione depletion in the subcellular environment surrounding an internalized nanocarrier or components thereof could be used to increase drug effectiveness.

If each cell contains approximately 5 millimolar GSH (Valko), and has a diameter of 10⁻⁵ meters and volume about 10⁻¹⁵ cubic meters, then each cell has about 3×10⁹ molecules of GSH (10⁻¹² L). Since each molecule of dehydroascorbic acid imported uses one GSH molecule, each particle could deplete ˜10⁵ to 10⁶ molecules of GSH. This is not sufficient for depletion of GSH from an entire cell, but it could produce significant localized depletion of GSH that could promote drug activity as well as peroxide activity.

The ascorbate in the surface of the nanocarrier of this invention can enhance the activities of various anti-cancer agents. Numerous chemotherapeutic agents are known to be made more effective by the presence of peroxide or the depletion of glutathione. Glutathione is used in the detoxification of anti-cancer agents including arsenic trioxide and peroxide by tumor cells. Glutathione depletion in vivo potentiates the anti-tumor activity of doxorubicin through inhibition of the multi-drug resistance associated protein that would otherwise expel doxorubicin from the cells. Some of the anti-cancer drugs that are expelled from tumor cells in a glutathione-dependant manner include the vinca alkaloids, anthracyclines, vincristine , and daunorubicin. A localized depletion of glutathione caused by internalization of DHAA on a carrier particle could allow higher activity of drug cargo by inhibiting the expulsion of the drug from the cell.

Peroxide generated from the ascorbate in the surface of the nanocarrier of this invention can enhance anti-cancer drug activity. Peroxide,is believed by many to potentiates the activity of antineoplastic quinones such as doxorubicin, mitomycin C, and diaziridinyl benzoquinone. The activity of arsenic trioxide is enhanced by depletion of glutathione and by peroxide. Motexafin gadolinium is believed to act in part through ROS generation. Extracellular ascorbic acid has been implicated in the activity of this drug. Effectiveness of MGd plus ascorbate was greater than the sum of the cytotoxicities of the individual components separately. As is evident even from this brief set of examples, the ascorbate in the surface of the nanocarrier of this invention will be capable of enhancing the effects of numerous anti-cancer agents.

The nanocarriers may be used in combination with other anti-cancer treatments. Multiple particle types can be combined for improved effectiveness. Co-therapies using diverse combinations of treatments would possible, with possible increased effectiveness-to-toxicity profiles.

Administration of the compositions of the present invention is preferably intravenous. It may also be oral, parenteral, through the mucosa, or transdermal.

The preferred embodiments herein described are not intended to be exhaustive or to limit the scope of the composition and method of the invention to the precise forms disclosed. They are chosen and described to best explain the principles of that invention and its application and practical use to allow others skilled in the art to understand its teachings.

### EXAMPLES

Liopsome Preparation

Liposomes containing palmitoyl ascorbate were generated. Palmitoyl ascorbate, egg phosphatidyl choline, and cholesterol solutions were combined. Paclitaxel was added to appropriate preparations. Wide ranges of palmitoyl ascorbate incorporation were easily attainable. Polymer-linked ascorbate (ascorbate-PEG-DSPE) was successfully incorporated in some preparations. A lipid film was formed following solvent evaporation. The lipid film was rehydrated in phosphate-buffered saline (PBS) to a final lipid concentration of 10 mg/m. The preparation was sonicated, then extruded through a membrane of 100 nm pore size. Liposomes were characterized for size and zeta-potential using a Beckman coulter N4 Plus particle sizer and a Brookhaven Zeta Sizer, respectively.

Micelle Preparation

Micelles were prepared from PEG-PE 2000 polymer and incorporating palmitoyl ascorbate or ascorbate-PEG-DSPE. Micelles can be generated through formation of a thin film for rehydration, as for liposome preparation. Alternatively, dry powders of components are sonicated in water, then dialyzed. Micelles can also be generated by dissolving amphipathic poymer in water-miscible solvent, then dialyzing.

Cell Death Analysis

Cells from various cancer and transformed cell lines were grown in 96 well plates to 40-50% confluence. Cell lines used inclused murine RAG mus musculus (Balb/c strain) renal adenocarcinoma, human ACHN kidney reneal cell adenocarcinoma, murine RENCA renal carcinoma cell; they may also include murine NIH/3T3 fibroblasts and drug-sensitive EL4 T lymphoma and Lewis lung carcinoma cells; human drug-sensitive NCI-H82 small cell lung carcinoma, COLO205 colorectal adenocarcinoma, MCF7 breast adenocarcinoma, and A2780 ovarian carcinoma cells; and human MDR A2780/ADR ovarian carcinoma cells. Cells were treated with appropriate liposomes for 1 hour, then washed. Cells were then incubated for 24 hours in complete cell culture medium. The cell viability was then determined using a standard methyl terazolium salt (MTS) assay, which produces a measured color change.

Cancer-Specific Cell Association Study

To evaluate the cell binding, a co-culture model was used with fluorescently labeled liposomes, the the results analyzed by flow cytometry. Palmitoyl ascorbate liposomes were fluorescently labeled with 0.5% rhodamine. Mouse embryo yolk sac cells expressing the green fluorescent protein GFP were co-cultured in flasks with various tumor cell types at a 1:1 ratio. Cell cultures were treated with 200 μl of liposome preparation in 5 ml of medium and incubated for 1 hour. Cells were then removed from the flasks using trypsin and fixed through resuspension in 800 μl of 10% paraformaldehyde in PBS. The fixed cells were then analyzed on a BD FACS Calibur Flow Cytometer. The change in red fluorescence in the two cell populations was measured and the resulting differences plotted on a graph. Data shown represent 3 separate experiments.

### DESCRIPTION OF THE FIGURES

FIG. 1 is a graph of data showing increasing cell death on the vertical axis and cancer cells and transformed cells labeled on the horizontal axis. Palmitoyl ascorbate liposomes (2 millimolar palmitoyl ascorbate) cause death of multiple cancer cell lines.

FIG. 2 is a graph having percent cell death on the vertical axis and having increasing concentrations of pamitoyl ascorbate incorporation into liposomes on the horizontal axis. Increasing concentrations of palmitoyl ascorbate in the liposome formulations are increasingly toxic to MCF7 cancer cells. Micelle formulation formulated from PEG2000 and palmitoyl ascorbate show high toxicity to MCF7 cancer cells even at very low palmitoyl ascorbate concentrations.

FIG. 3 is a graph having percent of cells in the assay associated with rhodamine-labeled palmitoyl ascorbate liposomes. Percentages for non-cancerous, green fluorescent control cells are shown in the red bars, and percentages for cancerous cells are shown in the blue bars. Standard deviation for all samples was below 5% except 3T3 liposome-treated cells which had a standard deviation of 11.5%

FIG. 4 is a fluorescent microscope image showing RAG tumor cells associating with rhodamine-labeled palmitoyl ascorbate liposomes. The cells shown on the right were treated with tumor necrosis factor (TNF) alpha during PA liposome treatment. The cells on the left were not treated with TNF during PA liposome treatment.

FIG. 5 is a graph showing percent death of MCF7 cancer cells on the vertical axis and labels of liposome treatments on the horizontal axis. Liposomes incorporating palmitoyl ascorbate are more toxic to cancer cells than plain liposomes. Palmitoyl ascorbate liposomes loaded with paclitaxel are more toxic to cancer cells than plain liposomes loaded with paclitaxel. Ascorbic acid added to the treatment did not enhance the toxicity of paclitaxel in plain liposomes.

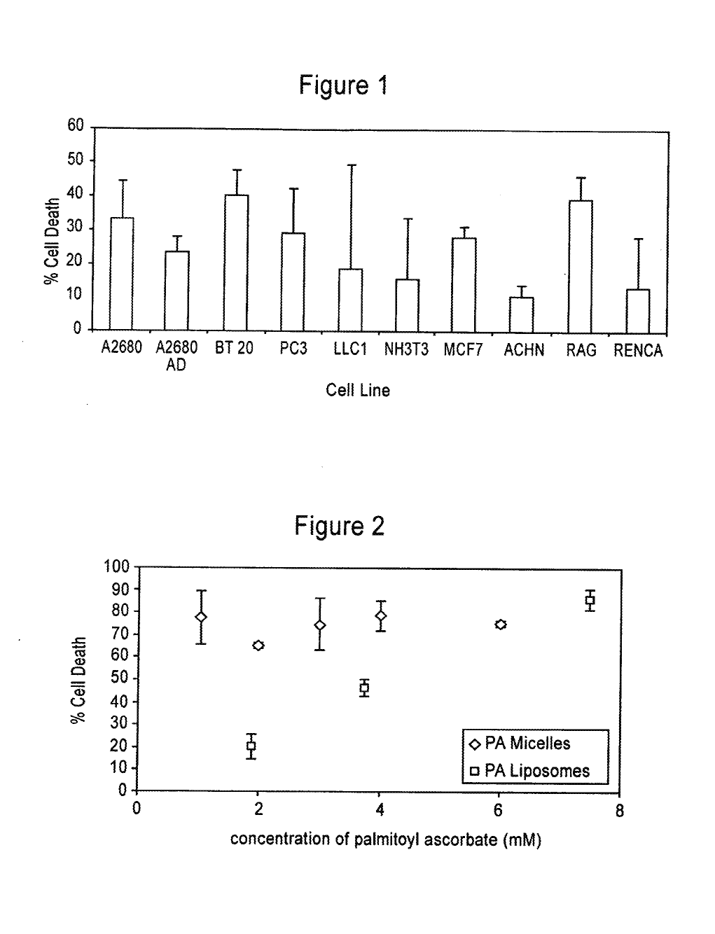

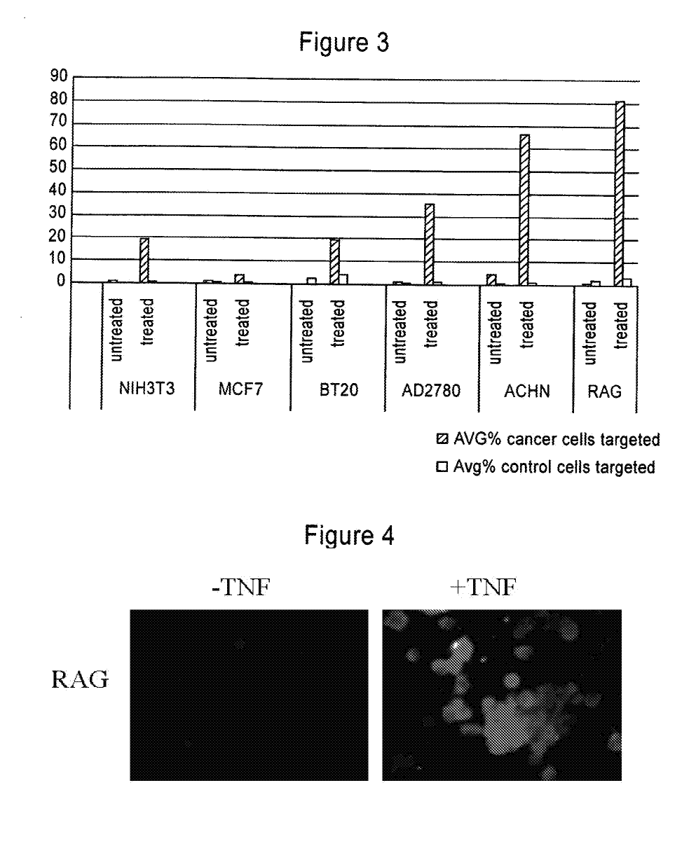

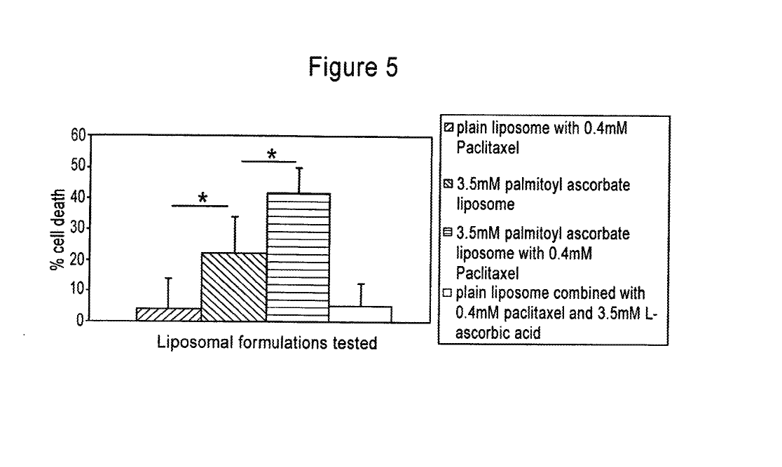

1. A method for treating cancer or delaying the progression of a cancer in an animal or a human comprising:
administering to the animal or the human having a cancer a composition in an amount effective to treat cancer or delay the progression of cancer in the animal or the human,
wherein the composition comprises a pharmaceutically acceptable carrier structure and ascorbate which is joined to the carrier structure

2. The method of claim 1,
wherein the ascorbate is incorporated in the surface of the carrier structure, and
wherein the carrier structure is a nanoscale drug delivery nanocarrier.

3. The method of claim 1, wherein the administering of the composition is intravenous.

4. The method of claim 1, wherein the administering of the composition is oral.

5. The method of claim 1, wherein the administering of the composition is transdermal.

6. The method of claim 1, wherein the administering of the composition is by injection or local infusion.

7. The method of claim 1, wherein the cancer is selected from the group consisting of Hodgkin's Disease, Non-Hodgkin's Lymphoma, neuroblastoma, breast cancer, ovarian cancer, lung cancer, renal cell carcinoma, rhabdomyosarcoma, primary thrombocytosis, primary macroglobulinemia, small-cell lung tumors, brain tumors, stomach cancer, kidney cancer, bone cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

8. The method of claim 1, wherein the cancer has a hypoxic gene expression pattern.

9. The method of claim 1, wherein said pharmaceutically acceptable carrier structure contains an anti-cancer active agent selected from the group consisting of 5-fluoropyrimidines, anti-angiogenics, antimicrotubule agents, cytidine analogs, alkylating agents, anticancer antibiotics, ascorbate or a derivative thereof, bisphosphonates, bleomycin, cisplatin and analogs, cytidine analogues, heat-generating substances, hydroxyurea, immune response modifiers, nucleic acids and analogues, magnetic oscillation substrates, mTOR inhibitors, purine anti-metabolites, radioactive agents, radiation response modifiers, differentiation-inducing agents, thalidomide, lenalidomide, an antineoplastic quinine, paclitaxel, topoisomerase inhibitors, and tyrosine kinase signal inhibitors.

10. The method of claim 1, wherein the carrier structure contains an imaging agent .

11. The method of claim 9, wherein the anti-cancer active agent is thalidomide, lenalidomide or an analogue thereof.

12. The method of claim 9, wherein the anti-cancer active agent is the radioactive agent.

13. The method of claim 9, wherein the anti-cancer active agent is DNA, RNA, miRNA, or a synthetic nucleotide polymer compound.

14. The method of claim 9, wherein the anti-cancer active agent is the ascorbate or derivative thereof.

15. The method of claim 9, wherein the anti-cancer active agent is the antineoplastic quinine

16. The method of claim 9, wherein the anti-cancer active agent is arsenic trioxide.

17. A composition for treating cancer, comprising:
a pharmaceutically acceptable carrier structure and ascorbate which is joined to the carrier structure,
wherein said pharmaceutically acceptable carrier structure contains an anti-cancer active agent selected from the group consisting of 5-fluoropyrimidines, anti-angiogenics, antimicrotubule agents, cytidine analogs, alkylating agents, anticancer antibiotics, ascorbate or a derivative thereof, bisphosphonates, bleomycin, cisplatin and analogs, cytidine analogues, heat-generating substances, hydroxyurea, immune response modifiers, nucleic acids and analogues, magnetic oscillation substrates, mTOR inhibitors, purine anti-metabolites, radioactive agents, radiation response modifiers, differentiation-inducing agents, thalidomide, lenalidomide, an antineoplastic quinine, paclitaxel, topoisomerase inhibitors, and tyrosine kinase signal inhibitors.

18. The composition of claim 17, wherein the ascorbate is linked to the carrier structure through an ascorbate C6 and/or C2 position to a lipid, polymer or other component of the carrier structure.

19. The composition of claim 17,
wherein the ascorbate is incorporated into the nanocarrier as a lipid-linked component.

20. The composition of claim 17, wherein the ascorbate is linked to polyethylene glycol (PEG), another biocompatible polymer or another polymer block.

21. The composition of claim 17, wherein the carrier structure is a biocompatible delivery system 1-1000 nanometers in diameter.

22. The composition of claim 17, wherein the carrier structure sensitive to modification by reactive oxygen species including peroxide and superoxide.

23. The composition of claim 22, wherein the carrier structure includes poly(propylene sulfide) blocks or peroxide-sensitive lipids.

24. The composition of claim 17, wherein the carrier structure is a liposome capable of being loaded with the anti-cancer active agent.

25. The composition of claim 17, wherein the carrier structure is a nanoparticle, polymersome, bi-block and tri-block polymersome, aptamer, dendrimer or polymer-stabilized liposome.

26. The composition of claim 17, wherein the carrier structure is a micelle.

27. The composition of claim 26, wherein the carrier structure is a worm-like micelle.

28. The composition of claim 17, wherein the carrier structure is a nano-shell structure.

29. The composition of claim 17, wherein the ascorbate is linked to the carrier structure by a spacer or linker.

30. The composition of claim 29, wherein the linker is a hydrocarbon linker of 1-30 carbon units in length.

31. The composition of claim 17, wherein the carrier structure is sensitive to low pH within tumors.

32. The composition of claim 31, wherein the carrier structure includes a member selected from the group consisting of poly (Beta-Amino Ester), poly (L-histidine), poly(DL lactide), poly(vinyl alcohol), N-isopropylacrylamide, and polyacrylamide.

33. The composition of claim 17, wherein the carrier structure is sensitive low pH within endosomes of cells.

34. The composition of claim 33, wherein the carrier structure includes a member selected from poly(L-lactide), polycaprolactone , poly(Beta-Amino Ester), polylactic acid, poly(DL lactide), poly(Beta-Amino Ester), poly (L-histidine), poly(vinyl alcohol), N-isopropylacrylamide, and polyacrylamide.

35. The composition of claim 28, wherein the nano-shell structure comprises a core comprising the anti-cancer active agent, an intermediate layer surrounding the core, and an outer layer surrounding the intermediate layer, said outer layer being capable of dissolving in an acidic environment.

36. The composition of claim 35, wherein the inner layer is hydrophobic and the outer layer is hydrophilic.

37. The composition of claim 36, wherein the hydrophobic inner layer comprises a pharmaceutical agent.

38. The composition of claim 17, wherein the carrier structure includes dimethyl maleic anhydride, cis-aconityl, or hydrazone linkages.

39. The composition of claim 17, wherein the carrier structure contains a peptide sequence which is cleavable by one or more proteases.

40. The composition of claim 17, wherein the carrier structure contains the amino acid residue sequence glycine-phenylalanine-leucine-glycine.

41. The composition of claim 17, wherein the carrier structure contains cationic peptide sequences.

42. The composition of claim 17, wherein the carrier structure contains intracellular localization signals.

43. The composition of claim 17, wherein the carrier structure includes cationic cell penetrating peptides.

44. The composition of claim 17,
wherein the carrier structure is a biocompatible polymersome vesicle consisting essentially of a semi-permeable, thin-walled encapsulating membrane, capable of encapsulating least one encapsulant therein,
wherein the membrane is formed in an aqueous solution without an organic solvent,
wherein the membrane comprises one or more wholly synthetic, super-amphiphilic molecules that are polymeric and self-assemble directly into the vesicle due to amphilicity, without post-assembly polymerization, and
wherein at least one super-amphiphile molecule is a block copolymer.

45. The composition of claim 17,
wherein the carrier structure is a solid nano-sphere encapsulated in a pH sensitive or salt sensitive micro-sphere, said pH sensitive or salt sensitive micro-sphere being formed of a pH sensitive or salt sensitive matrix material, and
wherein the anti-cancer active agent is incorporated into said solid nano-sphere or said microsphere or in both said solid nano-sphere and said micro-sphere.

46. The composition of claim 17,
wherein the carrier structure is a worm-like micelle comprising one or more wholly synthetic, polymeric, super-amphiphilic molecules that self assemble in aqueous solution, without organic solvent or post assembly polymerization, and
wherein at least one of said super-amphiphilic molecules is a hydrophilic block copolymer, the weight fraction of which, relative to total copolymer molecular weight, directs assembly of the amphiphilic molecules into the worm-like micelle of up to one or more microns in length, and determines its stability, flexibility and convective responsiveness.

47. The composition of claim 17, wherein the carrier structure is a vesicle formed from a lipid or a mixture of lipids selected from the group consisting of phosphatidylcholines, phosphatidylethanolamines, phosphatidic acids, phosphatidylserines, phosphatidylglycerols, cardiolipins, poly(ethylene glycol) lipid conjugates, sphingomyelins, cationic lipids, trioctanoin, triolein, dioctanoyl glycerol, cholesterol, and dioleoyl-glutaric acid.

48. The composition of claim 17, wherein the carrier structure is formed from a plurality of di-block copolymers, tri-block polymers, or mixtures thereof.

49. The composition of claim 17, wherein the carrier structure is a vesicle having dimensions of less than 10 microns.

50. The composition of claim 17,
wherein the carrier structure is worm-like micelle which comprises one or more amphiphilic block copolymers capable of self assembly in aqueous solution,
wherein the amphiphilic block copolymer comprises at least one hydrophilic block and at least one hydrophobic block, the at least one hydrophobic block being hydrolytically unstable in the pH range of about 5 to about 7,
wherein the at least one hydrophobic block degrades in the micelle at a rate which controls the rate of hydrolysis of the worm-like micelle; and
wherein said hydrophobic block decomposes at a known rate based on a known pH, thereby releasing said anti-cancer active agent.

51. The method of claim 1, wherein said pharmaceutically acceptable carrier structure contains sunitinib and the cancer is a gastrointestinal stromal tumor.

52. The method claim 1, wherein two or more types of compositions including the ascorbate are co-administered.

53. The method of claim 52, wherein anti-cancer active agents carried within the carrier structures of the two or more types of compositions including the ascorbate are capable of acting together.

54. The method of claim 52, wherein the two or more types of compositions including the ascorbate have distinct delivery profiles.

55. The method of claim 1, wherein said pharmaceutically acceptable carrier structure contains an anti-cancer active agent, and
wherein said carrier structure is capable of releasing the anti-cancer active agent in the presence of a reactive oxygen species.

56. The method of claim 55,
wherein the ascorbate is incorporated in the surface of the carrier structure, and
wherein the carrier structure is a nanoscale drug delivery nanocarrier.

57. The method of claim 55, wherein the lipid-linked component is ascorbic acid 6-palmitate.

58. The method of claim 55, wherein the carrier structure includes poly(propylene sulfide) blocks or peroxide-sensitive lipids.

59. The method of claim 55, wherein the carrier structure is a liposome.

60. The method of claim 55, wherein the carrier structure is a nanoparticle, polymersome, bi-block and tri-block polymersome, aptamer, dendrimer or polymer-stabilized liposome.

61. The method of claim 55, wherein the carrier structure is a micelle.

62. The method of claim 55, wherein the carrier structure is a worm-like micelle.

63. The method of claim 55, wherein the carrier structure is a nano-shell structure.

64. The method of claim 9, wherein the anti-cancer agent is paclitaxel.

65. The method of claim 9, wherein the carrier structure is sensitive to low pH within tumors.

66. The method of claim 9, wherein the carrier structure includes a member selected from the group consisting of poly (Beta-Amino Ester), poly (L-histidine), poly(DL lactide), poly(vinyl alcohol), N-isopropylacrylamide, and polyacrylamide.

67. The method of claim 9, wherein the carrier structure is sensitive to low pH within endosomes of cells.

68. The composition of claim 17, wherein said carrier structure is capable of releasing the anti-cancer active agent in the presence of a reactive oxygen species.

69. The composition of claim 17, wherein the carrier structure contains polyethylene glycol (PEG).

70. A method of visualizing tumors in an animal or a human, comprising:
administering to the animal or the human a composition in an amount effective to visualize a tumor in the animal or the human,
wherein the composition comprises a pharmaceutically acceptable carrier structure containing an imaging agent or a radioactive agent and ascorbate which is joined to the carrier structure, and
wherein the carrier structure is capable of localizing to cancer cells.

71. The method of claim 70, wherein the carrier structure includes poly(propylene sulfide block or peroxide-sensitive lipids.

72. The method of claim 70, wherein the composition comprises the imaging agent.

73. The method of claim 70, wherein the composition comprises the radioactive agent.

74. The method of claim 71, wherein the composition comprises the imaging agent.

75. The method of claim 71, wherein the composition comprises the radioactive agent.

76. The method of claim 70, wherein the composition activates or releases the imaging agent near cancer cells.

77. The method of claim 70, wherein the composition activates or releases the imaging agent in tumor tissues.

78. The method of claim 70, wherein the composition activates or releases the imaging agent in the presence of hypoxia.

79. The method of claim 70, wherein the composition activates the imaging agent in the presence of a low pH.

80. The method of claim 70, wherein the composition activates the imaging agent in the presence of a reactive oxygen species.

81. The method of claim 70, wherein the composition activates the imaging agent in the presence of an increased accumulation of said carrier structure.
